(19) United States
(12) Patent Application Publication
(10) Pub. No.: US 2002/0068712 A1
(43) Pub. Date: Jun. 6, 2002

(54) Use of decreasing levels of functional transient receptor potential gene product

(76) Inventor: Troy Stevens, Daphne, AL (US)

Correspondence Address: Braman & Rogalskyj, LLP, P.O. Box 352, Canandaigua, NY, 14424-0352, US

(21) Appl. No.: 09/840,298

(22) Filed: Apr. 23, 2001

Related U.S. Application Data
(63) Continuation of application No. 09/360,396, filed on Jul. 23, 1999.

(51) Int. Cl. A61K 48/00; C07H 21/04; C12N 15/87

(52) U.S. Cl. 514/44; 435/455; 536/23.2

(57) ABSTRACT

The invention provides a method of decreasing inflammatory gaps in pulmonary endothelial cells, the method comprising decreasing levels of functional transient receptor potential gene product in the cells. The invention further provides a method of treating or preventing an inflammatory condition in a subject, the method comprising administering to the subject an amount of a compound effective to decrease levels of functional transient receptor potential gene product in the cells of the subject.

This application claims priority of U.S. Provisional Patent Application No. 60/093,968, filed Jul. 24, 1998.

This invention was made with support from the United States Government under Grant Nos. HL-56050 and HL-60024 of the National Institutes of Health. The U.S. Government may have certain rights in this invention.

### FIELD OF THE INVENTION

The subject invention is directed generally to a method for treating or preventing an inflammatory condition, by decreasing inflammatory gaps in pulmonary endothelial cells, and more particularly to decreasing levels of functional transient receptor potential gene product.

### BACKGROUND OF THE INVENTION

Throughout this application various publications are referenced, many in parenthesis. Full citations for each of these publications are provided at the end of the Detailed Description. The disclosures of each of these publications in their entireties are hereby incorporated by reference in this application.

Pulmonary endothelial cells are a nonexcitable cell type in which humoral and neural signaling agents increase the free cytosolic Ca²⁺ concentration ([Ca²⁺]ᵢ) by inducing Ca²⁺ release from intracellular stores and Ca²⁺ entry across the cell membrane (4,34). Increased [Ca²⁺]ᵢ has been implicated in many endothelial-directed vascular responses including regulation of vascular tone and permeability (2, 23, 36), angiogenesis (20), and leukocyte trafficking (17). Activation of Ca²⁺ entry appears essential for each of these processes, although many modes of Ca²⁺ entry exist and a specific pathway regulating endothelial cell shape has yet to be identified.

It is widely accepted that endothelial cells possess capacitative, or store-operated, Ca²⁻ entry pathways (8, 13, 31, 35, 41, 42). However, specific store-operated Ca²⁺ channels (SOCs) responsible for Ca²⁺ entry into nonexcitable cell types are largely unidentified. Recent cloning and expression of the transient receptor potential (trp) gene product from the Drosophila melanogaster retina reveal that this product forms a Ca²⁺-permeant cation channel that mediates Ca²⁺ entry after intracellular inositol 1,4,5-trisphosphate [Ins(1,4,5)P₃] is generated and Ca²⁺ is liberated from intracellular stores (11, 15, 25). Six mammalian homologues of Drosophila Trp are known (5), and mRNAs for these have been reported in bovine aortic endothelial cells (12).

Non-cardiogenic pulmonary edema(s) represent a significant clinical complication that increases patient morbidity and mortality. Adult respiratory distress syndrome (ARDS) is the most severe form of these diseases and impacts conservatively 150,000 patients annually. Non-septic ARDS has an estimated mortality rate of 40-60% whereas septic ARDS has an estimated mortality rate exceeding 90%. Clinical management of ARDS patients is supportive, and unfortunately not a single pharmacologic strategy has been utilized successfully to improve patient outcome. The paucity of effective drug therapy and poor prognosis in these patients indicates the mechanisms underlying inception, propagation and resolution of the disease are not well understood.

Since the formation of inflammatory gaps in pulmonary endothelial cells is characteristic of inflammation, any methods that can decrease these inflammatory gaps could be useful in treating and/or preventing inflammation.

### SUMMARY OF THE INVENTION

The subject invention provides such a method which involves the transient receptor potential (trp) gene. More particularly, the invention provides a method of decreasing inflammatory gaps in pulmonary endothelial cells, the method comprising decreasing levels of functional transient receptor potential gene product (TRP) in the cells. The invention further provides a method of treating or preventing an inflammatory condition in a subject, the method comprising administering to the subject an amount of a compound effective to decrease levels of functional transient receptor potential gene product in the cells of the subject.

### BRIEF DESCRIPTION OF THE DRAWINGS

These and other features and advantages of this invention will be evident from the following detailed description of preferred embodiments when read in conjunction with the accompanying drawings in which:

FIGS. 1A-1C illustrate store-operated Ca²⁺ entry in rat (R) pulmonary arterial endothelial cells (PAECs). 1A: individual traces from cells challenged with thapsigargin (TG; 1 μM) in presence of 2 mM extracellular Ca²⁺ concentration ([Ca²⁺]ₒ). 1B: representative trances comparing TG-induced cytosolic Ca²⁺ concentration ([Ca²⁺]ᵢ) response in presence (solid line0 and absence (dashed line) of [Ca²⁺]ₒ. [Ca²⁺]ₒ was increased from 100 nM to 2 mM at time indicated by Ca²⁺. Arrows, time of addition. 1C: averaged data from all experiments conducted in presence (η=5 cells; open bars) and absence (η=5 cells; solid bars) of [Ca²⁺]ₒ t, Time. *Significant difference compared with respective baseline fluorescence ratio of Ca²⁺-bound (340-nm) to Ca²⁺-unbound (380-nm) excitation wavelengths emitted at 510 nm (340/380), P<0.05;

FIGS. 2A-2B illustrate currents in RPAECs which were measured under whole cell configuration with bath and pipette solutions given in METHODS. Average (±SE) currents were calculated from last 20 ms of each 200-ms voltage step. 2A: summary of current voltage recordings for unstimulated (η=4) and thapsigargin-treated (1 μM thapsigargin in pipette; η=8) RPAECs normalized to membrane capacitance to yield current density. Insets: sample sets of current pulses. 2B: net inward current density generated in response to thapsigargin;

FIGS. 3A-3D illustrate scanning electron micrographs of RPAEC monolayers (×2,000). 3A: control (2 mM [Ca²⁺]ₒ). 3B: thapsigargin treatment. 3C: RPAECs in 100 nM [Ca²⁺]ₒ challenged with thapsigargin. 3D: readdition of 2 mM [Ca²⁺]ₒ to RPAECs treated as in C;

FIGS. 4A-4B illustrate the effect of [Ca²⁺]ₒ on F-actin distribution. Confocal microscopy was performed on 0.3-μm sections. Three micrographs/treatment are shown. 4A: unchallenged RPAEC monolayers in 2 mM [Ca²⁺]ₒ. 4B: RPAECs incubated in 100 nM [Ca²⁺]ₒ. 1: cross section through tops of cells. In 2 mM [Ca²⁺]ₒ staining appeared as a peripheral band with apparent cell-to-cell contact sites. In low [Ca²⁺]ₒ, diffuse punctate staining was observed throughout cells, but contact sites between cells were still obvious. II and III: cross sections through middle and lower aspects of cells, respectively. In 2 mM [Ca²⁺]ₒ, F-actin aligned in radiating strands, with obvious F-actin-containing focal contact sites. Low [Ca²⁺]ₒ was characterized by diffuse staining throughout, with cell junction integrity still intact;

FIGS. 5A-5C illustrate the ffect of thapsigargin on F-actin organization in presence of 2 mM (5A), 100 nM (5B), and 100 nM [Ca²⁺]ₒ followed by restoration of [Ca²⁺]ₒ to 2 mM (5C). Confocal microscopy was performed on 0.3-μm sections. Three micrographs/treatment are shown. I: cross section through upper portion of cells. In presence of 2 mM [Ca²⁺]ₒ, diffuse perinuclear staining is evident. In low [Ca²⁺]ₒ, a peripheral actin band with cell-to-cell contact sites is prominent. This peripheral band retracted after [Ca²⁺]ₒ was readded, and intercellular actin projections are discernible. II and III: cross sections of middle and lower portions of cells, respectively. In presence of 2 mM [Ca²⁺]ₒ, peripheral (cortical) actin band is absent, and F-actin appears to align in stress fibers. In low [Ca²⁺]ₒ, diffuse punctate staining is observed, but cortical actin band is still present. On readdition of [Ca²⁺]ₒ, stress fiber formation is obvious.

### DETAILED DESCRIPTION OF THE INVENTION

The subject invention is based on the discovery that decreasing levels of functional transient receptor potential gene product (TRP) in a cell (such as by decreasing transient receptor potential (trp) gene expression or by decreasing activity of TRP or by decreasing formation of calcium channels by TRP) can decrease inflammatory gaps in pulmonary endothelial cells. Inflammatory gaps occur between pulmonary endothelial cells due to an increase in intracellular calcium in the cells. The increase in intracellular calcium in the cells occurs via TRP calcium channels. Decreasing levels of functional TRP can therefore be used to decrease intracellular calcium and therefore decrease gap formation and therefore decrease inflammation.

TRPs form calcium channels which belong to the family of store-activated calcium channels. One or more isoforms of TRP may be required to form a functional calcium channel. Decreasing “levels” of functional TRP refers to decreasing expression of the trp gene, decreasing activity of the TRP such as by inhibiting one or more TRP isoforms, and/or decreasing the formation of active membrane-spanning calcium channels by the TRP.

The invention thus provides a method of decreasing inflammatory gaps in pulmonary endothelial cells, the method comprising decreasing levels of TRP in the cells.

Levels of TRP in the cells can be decreased by various methods, at the gene and protein and “functional calcium channel” levels. In one embodiment, the levels are decreased by decreasing trp gene expression of the TRP in the cells. This can be accomplished by exposing the cells to a compound which decreases trp gene expression of the TRP. The compound could be, for example, an antisense oligonucleotide targeted to the trp gene.

In a similar embodiment, the compound which decreases trp gene expression of the TRP could be a ribozyme, which is a special category of antisense RNA molecule having a recognition sequence complementary to the mRNA encoding the TRP. A ribozyme not only complexes with a target sequence via complementary antisense sequences, but also catalyzes the hydrolysis, or cleavage, of the template mRNA molecule. The expression of the TRP protein is therefore prevented.

Other methods for decreasing trp gene expression could also involve site-directed mutagenesis of the trp gene to prevent expression of the TRP, or various gene therapy techniques.

Levels, in particular activity, of TRP in the cell can also be decreased by exposing the cells to an inhibitor of the TRP. Currently known inhibitors of voltage gated calcium channels include, for example, nifedipine, nitrendipine, verapamil, and related compounds. Other inhibitors of the TRP could also readily be identified by various screening methods used in the art (see more detailed discussion below). In addition to chemical inhibitors, peptide inhibitors could also be identified with currently known screening methods (for example, using phage display libraries and other peptide screening methods).

Levels of TRP in the cell can also be decreased by exposing the cells to a compound which interferes with membrane calcium channel formation by the TRP.

Since the method of the subject invention is a method of decreasing inflammatory gaps in pulmonary endothelial cells, the cells of interest can be of human or animal origin.

The invention further provides a method of treating or preventing an inflammatory condition in a subject, the method comprising administering to the subject an amount of a compound effective to decrease levels of TRP in the cells of the subject. As above, the compound may decrease levels of TRP by decreasing trp gene expression of the TRP, or by inhibiting the TRP, or by interfering with membrane calcium channel formation by the TRP.

The method is useful in an inflammatory condition. Examples of inflammatory conditions include regional inflammatory disorders, such as asthma (late phase), pancreatitis, inflammatory bowel disease (IBD), peritonitis, rheumatoid arthritis, osteoarthritis, myocardial infarction, ocular inflammatory states, and stroke. Examples of inflammatory conditions also include systemic inflammatory disorders, such as systemic inflammatory response syndrome (SIRS), cardiogenic shock, adult respiratory distress syndrome (ARDS), multiple-organ dysfunction (MOD), septic shock, and infant respiratory distress syndrome (IRDS).

In one embodiment, the invention employs oligonucleotides targeted to nucleic acids encoding functional transient receptor potential gene product (TRP). The relationship between an oligonucleotide and its complementary nucleic acid target to which it hybridizes is commonly referred to as “antisense”. “Targeting” an oligonucleotide to a chosen nucleic acid target, in the context of this invention, is a multistep process. The process usually begins with identifying a nucleic acid sequence whose function is to be modulated. In the subject invention, this may be, for example, the cellular gene (or mRNA made from the gene) for trp; i.e., the target is a nucleic acid encoding TRP, the trp gene, or mRNA expressed from the trp gene. The targeting process also includes determination of a site or sites within the nucleic acid sequence for the oligonucleotide interaction to occur such that the desired effect, modulation of gene expression, will result. Once the target site or sites have been identified, oligonucleotides are chosen which are sufficiently complementary to the target, i.e., hybridize sufficiently well and with sufficient specificity, to give the desired modulation.

In the context of this invention “modulation” means either inhibition or stimulation. Inhibition of trp gene expression is presently the preferred form of modulation. This modulation can be measured in ways which are routine in the art, for example by Northern blot assay of mRNA expression or Western blot assay of protein expression. Effects on inflammatory gaps between cells can also be measured, as taught in the examples of the instant application. “Hybridization”, in the context of this invention, means hydrogen bonding, also known as Watson-Crick base pairing, between complementary bases, usually on opposite nucleic acid strands or two regions of a nucleic acid strand. Guanine and cytosine are examples of complementary bases which are known to form three hydrogen bonds between them. Adenine and thymine are examples of complementary bases which form two hydrogen bonds between them. “Specifically hybridizable” and “complementary” are terms which are used to indicate a sufficient degree of complementarity such that stable and specific binding occurs between the DNA or RNA target and the oligonucleotide. It is understood that an oligonucleotide need not be 100% complementary to its target nucleic acid sequence to be specifically hybridizable. An oligonucleotide is specifically hybridizable when binding of the oligonucleotide to the target interferes with the normal function of the target molecule to cause a loss of utility, and there is a sufficient degree of complementarity to avoid non-specific binding of the oligonucleotide to non-target sequences under conditions in which specific binding is desired, i.e., under physiological conditions in the case of in vivo assays or therapeutic treatment or, in the case of in vitro assays, under conditions in which the assays are conducted.

In various embodiments of this invention, oligonucleotides are provided which are targeted to mRNA encoding TRP. In accordance with this invention, persons of ordinary skill in the art will understand that mRNA includes not only the coding region which carries the information to encode a gene product using the three letter genetic code, including the translation start and stop codons, but also associated ribonucleotides which form a region known to such persons as the 5′-untranslated region, the 3′-untranslated region, the 5′ cap region, intron regions and intron/exon or splice junction ribonucleotides. Thus, oligonucleotides may be formulated in accordance with this invention which are targeted wholly or in part to these associated ribonucleotides as well as to the coding ribonucleotides. The functions of mRNA to be interfered with include all vital functions such as translocation of the RNA to the site for protein translation, actual translation of protein from the RNA, splicing or maturation of the RNA and possibly even independent catalytic activity which may be engaged in by the RNA. The overall effect of such interference with the RNA function is to cause interference with trp gene expression.

In the context of this invention, the term “oligonucleotide” refers to an oligomer or polymer of nucleotide or nucleoside monomers consisting of naturally occurring bases, sugars and intersugar (backbone) linkages. The term “oligonucleotide” also includes oligomers comprising non-naturally occurring monomers, or portions thereof, which function similarly. Such modified or substituted oligonucleotides are often preferred over native forms because of properties such as, for example, enhanced cellular uptake and increased stability in the presence of nucleases.

The compounds and/or inhibitors used in the methods of the subject invention encompass any pharmaceutically acceptable salts, esters, or salts of such esters, or any other compound/inhibitor which, upon administration to an animal including a human, is capable of providing (directly or indirectly) the biologically active metabolite or residue thereof. Accordingly, for example, the disclosure is also drawn to prodrugs and pharmaceutically acceptable salts of the compounds and/or inhibitors used in the subject invention, pharmaceutically acceptable salts of such prodrugs, and other bioequivalents.

In regard to prodrugs, the compounds and/or inhibitors for use in the invention may additionally or alternatively be prepared to be delivered in a prodrug form. The term prodrug indicates a therapeutic agent that is prepared in an inactive form that is converted to an active form (i.e., drug) within the body or cells thereof by the action of endogenous enzymes or other chemicals and/or conditions.

In regard to pharmaceutically acceptable salts, the term pharmaceutically acceptable salts refers to physiologically and pharmaceutically acceptable salts of the compounds and/or inhibitors used in the subject invention: i.e., salts that retain the desired biological activity of the parent compound and do not impart undesired toxicological effects thereto.

The oligonucleotides used in the method of the subject invention preferably are from about 8 to about 50 nucleotides in length. In the context of this invention it is understood that this encompasses non-naturally occurring oligomers, preferably having 8 to 50 monomers.

The oligonucleotides used in accordance with this invention may be conveniently and routinely made through the well-known technique of solid phase synthesis. Equipment for such synthesis is sold by several vendors including Applied Biosystems. Any other means for such synthesis may also be employed; the actual synthesis of the oligonucleotides is well within the skill of the art. It is also well known to use similar techniques to prepare other oligonucleotides such as the phosphorothioates and alkylated derivatives. It is also well known to use similar techniques and commercially available modified amidites and controlled-pore glass (CPG) products such as biotin, fluorescein, acridine or psoralen-modified amidites and/or CPG (available from Glen Research, Sterling, Va.) to synthesize fluorescently labeled, biotinylated or other modified oligonucleotides such as cholesterol-modified oligonucleotides.

In the context of this invention, to “expose” cells (including the cells of tissues) to a compound and/or inhibitor means to add the compound and/or inhibitor, usually in a liquid carrier, to a cell suspension or tissue sample, either in vitro or ex vivo, or to administer the compounds and/or inhibitor to cells or tissues within an animal (including a human) subject.

For therapeutics, methods of decreasing inflammatory gaps in pulmonary endothelial cells and methods of preventing and treating inflammatory conditions are provided. The formulation of therapeutic compositions and their subsequent administration is believed to be within the skill in the art. In general, for therapeutics, a patient suspected of needing such therapy is given a compound and/or inhibitor in accordance with the invention, commonly in a pharmaceutically acceptable carrier, in amounts and for periods which will vary depending upon the nature of the particular disease, its severity and the patient's overall condition. The pharmaceutical compositions may be administered in a number of ways depending upon whether local or systemic treatment is desired and upon the area to be treated. Administration may be topical (including ophthalmic, vaginal, rectal, intranasal, transdermal), oral or parenteral. Parenteral administration includes intravenous drip or infusion, subcutaneous, intraperitoneal or intramuscular injection, pulmonary administration, e.g., by inhalation or insufflation (especially relevant for treatment of asthma), or intrathecal or intraventricular administration.

Formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

Compositions for oral administration include powders or granules, suspensions or solutions in water or non-aqueous media, capsules, sachets or tablets. Thickeners, flavoring agents, diluents, emulsifiers, dispersing aids or binders may be desirable.

Compositions for parenteral, intrathecal or intraventricular administration may include sterile aqueous solutions which may also contain buffers, diluents and other suitable additives.

In addition to such pharmaceutical carriers, cationic lipids may be included in the formulation to facilitate oligonucleotide uptake. One such composition shown to facilitate uptake is Lipofectin (BRL, Bethesda Md.).

Dosing is dependent on severity and responsiveness of the condition to be treated, with course of treatment lasting from several days to several months or until a cure is effected or a diminution of disease state is achieved. Optimal dosing schedules can be calculated from measurements of drug accumulation in the body. Persons of ordinary skill can easily determine optimum dosages, dosing methodologies and repetition rates. Optimum dosages may vary depending on the relative potency of individual compounds and/or inhibitors, and can generally be calculated based on IC₅₀'s or EC₅₀'s in in vitro and in vivo animal studies. For example, given the molecular weight of compound (derived from oligonucleotide sequence and/or chemical structure) and an effective dose such as an IC₅₀, for example (derived experimentally), a dose in mg/kg is routinely calculated.

The nucleic acid and amino acid sequences of various transient receptor potential genes are known and readily available from GenBank and described in the literature. For example, see GenBank Accession No. NP 003295 which discloses the 759 amino acid sequence of the human transient receptor potential channel 1, GenBank Accession No. AAC16725 which discloses the 123 amino acid sequence of the rat transient receptor potential protein 1, GenBank Accession No. AAD22978 which discloses the 778 amino acid sequence of the African clawed frog cation channel TRP-1, GenBank Accession Nos. NM 003304 and X89066 which each disclose the 4085 bp sequence of the mRNA for human TRPC1 protein, GenBank Accession No. AF061873 which discloses the 369 bp partial sequence of the mRNA for rat trp1, GenBank Accession No. U40980 which discloses the 372 bp partial sequence of the mRNA for house mouse trp-related protein 1, and GenBank Accession No. X90696 which discloses the 304 bp sequence of the mRNA for the African clawed frog trp-like protein. See also, Wes et al., Proc Natl Acad Sci USA 92(21):9652-9656 (Oct. 10, 1995).

Given these sequences, one can design appropriate antisense molecules for use in the subject invention. Furthermore, by expressing the functional TRP calcium channel in a host cell, one can screen for suitable compounds and/or inhibitors for use in the subject invention. The function of the encoded calcium channel can be assayed according to methods known in the art, such as by analysis of the channel following the functional expression of the channel in oocytes of the frog Xenopus laevis. As used herein, “functional” expression refers to the synthesis and any necessary post-translational processing of a calcium channel molecule in a cell so that the channel is inserted properly in the cell membrane and is capable of conducting calcium ions in accordance with a store-activated channel.

More particularly, having known nucleic acid molecules encoding the TRP, a method for screening a chemical agent (compound or inhibitor) for the ability of the chemical agent to modify calcium channel function begins by introducing the nucleic acid molecule encoding the TRP into a host cell, and expressing the TRP encoded by the molecule in the host cell. The expression results in the functional expression of a TRP calcium channel in the membrane of the host cell. The cell is then exposed to a chemical agent and evaluated to determine if the chemical agent modifies the function of the TRP calcium channel. From this evaluation, chemical agents effective in altering the function of the sodium channel can be found and utilized in the methods of the subject invention.

Drugs, such as peptide drugs, which inhibit the TRP or which interfere with function TRP calcium channel formation can be made using various methods known in the art. Initially, a monoclonal antibody can be prepared which specifically hybridizes to the TRP, thereby interfering with activity and/or channel formation.

The monoclonal antibodies can be produced by hybridomas. A hybridoma is an immortalized cell line which is capable of secreting a specific monoclonal antibody.

In general, techniques for preparing polyclonal and monoclonal antibodies as well as hybridomas capable of producing the desired antibody are well known in the art (see Campbell, A. M., “Monoclonal Antibody Technology: Laboratory Techniques in Biochemistry and Molecular Biology”, Elsevier Science Publishers, Amsterdam, The Netherlands (1984); St. Groth, et al., J Immunol Methods 35:1-21 (1980)). Any animal (mouse, rabbit, etc.) which is known to produce antibodies can be immunized with the TRP (or an antigenic fragment thereof). Methods for immunization are well known in the art. Such methods include subcutaneous or intraperitoneal injection of the TRP. One skilled in the art will recognize that the amount of the TRP used for immunization will vary based on the animal which is immunized, the antigenicity of the TRP, and the site of injection.

The TRP which is used as an immunogen may be modified or administered in an adjuvant in order to increase the TRP's antigenicity. Methods of increasing the antigenicity of a protein are well known in the art and include, but are not limited to, coupling the antigen with a heterologous protein (such as a globulin or beta-galactosidase) or through the inclusion of an adjuvant during immunization.

For monoclonal antibodies, spleen cells from the immunized animals are removed, fused with myeloma cells, such as SP2/O—Ag 15 myeloma cells, and allowed to become monoclonal antibody producing hybridoma cells.

Any one of a number of methods well known in the art can be used to identify the hybridoma cell which produces an antibody with the desired characteristics. These include screening the hybridomas with an ELISA assay, western blot analysis, or radioimmunoassay (Lutz, et al., Exp Cell Res 175:109-124 (1988)).

Hybridomas secreting the desired antibodies are cloned and the class and subclass are determined using procedures known in the art (Campbell, A. M., “Monoclonal Antibody Technology: Laboratory Techniques in Biochemistry and Molecular Biology”, Elsevier Science Publishers, Amsterdam, The Netherlands (1984)).

For polyclonal antibodies, antibody containing antisera is isolated from the immunized animal and is screened for the presence of antibodies with the desired specificity using one of the above-described procedures.

Once a monoclonal antibody which specifically hydridizes to the TRP is identified, the monoclonal (which is itself a compound or inhibitor which can be used in the subject invention) can be used to identify peptides capable of mimicking the inhibitory activity of the monoclonal antibody. One such method utilizes the development of epitope libraries and biopanning of bacteriophage libraries. Briefly, attempts to define the binding sites for various monoclonal antibodies have led to the development of epitope libraries. Parmley and Smith developed a bacteriophage expression vector that could display foreign epitopes on its surface (Parmley, S. F. & Smith, G. P., Gene 73:305-318 (1988)). This vector could be used to construct large collections of bacteriophage which could include virtually all possible sequences of a short (e.g. six-amino-acid) peptide. They also developed biopanning, which is a method for affinity-purifying phage displaying foreign epitopes using a specific antibody (see Parmley, S. F. & Smith, G. P., Gene 73:305-318 (1988); Cwirla, S. E., et al., Proc Natl Acad Sci USA 87:6378-6382 (1990); Scott, J. K. & Smith, G. P., Science 249:386-390 (1990); Christian, R. B., et al., J Mol Biol 227:711-718 (1992); Smith, G. P. & Scott, J. K., Methods in Enzymology 217:228-257 (1993)).

After the development of epitope libraries, Smith et al. then suggested that it should be possible to use the bacteriophage expression vector and biopanning technique of Parmley and Smith to identify epitopes from all possible sequences of a given length. This led to the idea of identifying peptide ligands for antibodies by biopanning epitope libraries, which could then be used in vaccine design, epitope mapping, the identification of genes, and many other applications (Parmley, S. F. & Smith, G. P., Gene 73:305-318 (1988); Scott, J. K., Trends in Biochem Sci 17:241-245 (1992)).

Using epitope libraries and biopanning, researchers searching for epitope sequences found instead peptide sequences which mimicked the epitope, i.e., sequences which did not identify a continuous linear native sequence or necessarily occur at all within a natural protein sequence. These mimicking peptides are called mimotopes. In this manner, mimotopes of various binding sites/proteins have been found.

The sequences of these mimotopes, by definition, do not identify a continuous linear native sequence or necessarily occur in any way in a naturally-occurring molecule, i.e. a naturally occurring protein. The sequences of the mimotopes merely form a peptide which functionally mimics a binding site on a naturally-occurring protein.

Many of these mimotopes are short peptides. The availability of short peptides which can be readily synthesized in large amounts and which can mimic naturally-occurring sequences (i.e. binding sites) offers great potential application.

Using this technique, mimotopes to a monoclonal antibody that recognizes TRP can be identified. The sequences of these mimotopes represent short peptides which can then be used in various ways, for example as peptide drugs that bind to TRP and decrease the activity of TRP. Once the sequence of the mimotope is determined, the peptide drugs can be chemically synthesized.

The peptides for use in the subject invention can contain any naturally-occurring or non-naturally-occurring amino acids, including the D-form of the amino acids, amino acid derivatives and amino acid mimics, so long as the desired function and activity of the peptide is maintained. The choice of including an (L)- or a (D)-amino acid in the peptide depends, in part, on the desired characteristics of the peptide. For example, the incorporation of one or more (D)-amino acids can confer increased stability on a peptide and can allow a peptide to remain active in the body for an extended period of time. The incorporation of one or more (D)-amino acids can also increase or decrease the pharmacological activity of a peptide.

The peptide may also be cyclized, since cyclization may provide the peptide with superior properties over their linear counterparts.

Modifications to the peptide backbone and peptide bonds thereof are encompassed within the scope of amino acid mimic or mimetic. Such modifications can be made to the amino acid, derivative thereof, non-amino acid moiety or the peptide either before or after the amino acid, derivative thereof or non-amino acid moiety is incorporated into the peptide. What is critical is that such modifications mimic the peptide backbone and bonds which make up the same and have substantially the same spacial arrangement and distance as is typical for traditional peptide bonds and backbones. An example of one such modification is the reduction of the carbonyl(s) of the amide peptide backbone to an amine. A number of reagents are available and well known for the reduction of amides to amines such as those disclosed in Wann et al., JOC 46:257 (1981) and Raucher et al., Tetrahedron Lett 21:14061 (1980). An amino acid mimic is, therefore, an organic molecule that retains the similar amino acid pharmacophore groups as are present in the corresponding amino acid and which exhibits substantially the same spatial arrangement between functional groups.

The substitution of amino acids by non-naturally occurring amino acids and amino acid mimics as described above can enhance the overall activity or properties of an individual peptide thereof based on the modifications to the backbone or side chain functionalities. For example, these types of alterations can enhance the peptide's stability to enzymatic breakdown and increase biological activity. Modifications to the peptide backbone similarly can add stability and enhance activity.

One skilled in the art, using the identified sequences can easily synthesize the peptides for use in the invention. Standard procedures for preparing synthetic peptides are well known in the art. The novel peptides can be synthesized using: the solid phase peptide synthesis (SPPS) method of Merrifield, J Am Chem Soc 85:2149 (1964) or modifications of SPPS; or, the peptides can be synthesized using standard solution methods well known in the art (see, for example, Bodanzsky, “Principles of Peptide Synthesis”, 2d Ed., Springer-Verlag (1993)). Alternatively, simultaneous multiple peptide synthesis (SMPS) techniques well known in the art can be used. Peptides prepared by the method of Merrifield can be synthesized using an automated peptide synthesizer such as the Applied Biosystems 431A-01 Peptide Synthesizer (Mountain View, Calif.) or using the manual peptide synthesis technique described by Houghten, Proc Natl Acad Sci USA 82:5131 (1985).

Methods

Isolation of RPAECs.

Male Sprague-Dawley rats (CD strain, 350-400 g; Charles River) were euthanized by an intraperitoneal injection of 50 mg of pentobarbital sodium (Nembutal, Abbott Laboratories, Chicago, Ill.). After sternotomy, the heart and lungs were removed en bloc, and the pulmonary arterial segment between the heart and lung hili was dissected, split, and fixed onto a 35-mm plastic dish. Endothelial cells were obtained by gentle intimal scraping with a plastic cell lifter and were seeded into a 100-mm petri dish containing 10 ml of seeding medium (˜1:1 DMEM-Ham's F-12+10% fetal bovine serum) (37). Afer incubation for 1 wk (21% O₂-5% CO₂-74% N₂ at 37° C.), smooth muscle cell contaminants were marked and then removed by pipette aspiration. Cells were verified as endothelial by positive factor VIII staining and uptake of 1,1′-dioctadecyl-3,3,3′,3′-tetramethylindocarbocyanine perchlorate (DiI)-labeled acetylated low-density lipoprotein. When the primary culture reached confluence, cells were passaged by trypsin digestion into T-75 culture flasks (Corning), and standard tissue culture techniques were followed until the cells were ready for experimentation (passages 6-20).

[Ca²⁺]ᵢ Measurement by Fura 2 Fluorescence.

RPAECs were seeded onto chambered glass coverslips (Nalge Nunc, Naperville, Ill.) and grown to confluence. [Ca²⁺]ᵢ was estimated with the Ca²⁺-sensitive fluorophore fura 2-AM (Molecular Probes, Eugene, Oreg.) according to methods previously described by our laboratory (38). Because this is the first report of [Ca²⁺]ᵢ measurements in cultured RPAECS, a summary of the technique will be presented. RPAECs were washed with 2 ml of a HEPES (Fisher Scientific, Atlanta, Ga.)-buffered physiological salt solution (PSS) containing (in g/l) 6.9 NaCl, 0.35 KCl, 0.16 KH₂PO₄, 0.141 MgSO₄, 2.0 D-glucose, and 2.1 NaHCO₃. The loading solution (1 ml) consisted of PSS plus 3 μM fura 2-AM, 3 μl of a 10% pluronic acid solution, and 2 mM or 100 nM CaCl₂. RPAECs were fura loaded for 20 min in a CO₂ incubator at 37° C. After this loading period, the cells were again washed with PSS (2 ml) and treated with deesterification medium (PSS+2 mM or 100 nM CaCl₂) for an additional 20 min. After deesterification, [Ca²⁺]ᵢ was assessed with an Olympus 1×70 inverted microscope at ×400 with a xenon arc lamp photomultiplier system (Photon Technologies, Monmouth Junction, N.J.), and data were acquired and analyzed with PTI Felix software. Epifluorescence (signal averaged) was measured from three to four endothelial cells in a confluent monolayer, and the changes in [Ca²⁺]ᵢ are expressed as the fluorescence ratio of the Ca²⁺-bound (340-nm) to Ca²⁺-unbound (380-nm) excitation wavelengths emitted at 510 nm.

Electrophysiological determination of store-operated Ca²⁺ entry.

Whole cell patch clamp was utilized to measure transmembrane ion flux in thapsigargin-stimulated RPAECs. Confluent RPAECs were enzyme dispersed, seeded onto 35-mm plastic culture dishes, and then allowed to reattach for at least 24 h before patch-clamp experiments were performed. Single RPAECs exhibiting a flat, polyhedral morphology were studied. These cells were chosen for study because their morphology was consistent with RPAECs from a confluent monolayer. The extracellular and pipette solutions were composed of the following (in mM): 1) extracellular: 110 tetraethylammonium aspartate, 10 calcium aspartate, 10 HEPES, and 0.5 3,4-diaminopyridine; and 2) pipette: 130 N-mehyl-D-glucamine, 1.15 EGTA, 10 HEPES, 1 Ca(OH)₂, and 2 Mg²⁺-ATP. Both solutions were adjusted to 290-300 mosM with sucrose and pH 7.4 with methane sulfonic acid, and [Ca²⁺]ᵢ was estimated as 100 nM (10). The pipette resistance was 2-5 MΩ. Data were obtained with a HEKA EPC9 amplifier (Lambrecht/Pfaltz) and sampled on-line with Pulse+Pulsefit software (HEKA). All recordings were made at room temperature (22° C.). To generate current-voltage (I-V) relationships, voltage pulses were applied from −100 to +100 mV in 20-mV increments, with a 200-ms duration during each voltage step and a 2-s interval between steps. The holding potential between each step was 0 mV.

Assessment of Endothelial Cell Shape Change.

RPAECs were seeded onto 35-mm plastic culture dishes and grown to confluence. Growth medium was replaced with experimental buffer (same as that used for [Ca²⁺]ᵢ measurements but without fura 2), and the cells were subjected to one of the following protocols: 1) vehicle control (5 min) in 2 mM or 100 nM extracellular Ca²⁺ concentration ([Ca²⁺]ₒ); 2) thapsigargin (1 μM, 5 min) in 2 mM or 100 nM ([Ca²⁺]ₒ; or 3) thapsigargin in 100 nM [Ca²⁺]ₒ+readdition of 2 mM CaCl₂ (5 min). At the end of each experiment, the cell monolayers were fixed in 3% gluteraldehyde-PBS for 2 h. The cells were washed two times with 0.1 M cacodylate buffer, dehydrated by immersion in a series of ethanol dilutions, critical point dried in CO₂, and covered with 20 nm of gold. Specimens were viewed at 10 kW at a 15° inclination. Scanning electron micrographs were taken of representative areas in the monolayer by a pathologist blinded to the experimental protocols.

Assessment of Filamentous Actin Arrangement.

Experiments to determine filamentous actin (F-actin) arrangement were conducted in parallel with those assessing endothelial cell shape. RPAECs were seeded onto glass coverslips, and F-actin was stained with Oregon Green-phalloidin (Molecular Probes) with a standard fixation and staining protocol. Cells were analyzed by confocal microscopy (excitation at 496 nm and emmision at 520 nm). Micrographs were taken at multiple cellular depths (0.3-μm steps, 13-15 sections) and were used to deduce the microfilamentous cytoskeleton configurations of the cells.

Identification of Trp Gene Products in Pulmonary Endothelial Cells.

For RT-PCR cloning experiments, RPAECs and human (H) PAECs (Clonetics, San Diego, Calif.) were studied. Standard techniques for RT-PCR subcloning were followed. All chemical reagents used were of molecular biological grade. Briefly, total RNA was extracted from RPAECs and HPAECs grown to confluence in 75-cm² culture flasks (˜10⁷ cells) with RNA Stat-60 (Tel-Test “B”, Friendswood, Tex.). First-strand synthesis was performed with reverse transcriptase and oligo(dT) primer (GIECO BRL) on 1 μg of DNase I-treated total RNA. PCR was then performed with the following sets of primers: 1) Trp1: 5′-TCG CCG AAC GAG GTG ATG G-3′ (sense) and 5′-GTT ATG GTA ACA GCA TTT CTC C-3′ (antisense), 2) Trp3: 5′-ACC TCT CAG GCC TAA GGG AG-3′ (sense) and 5′-CCT TCT GAA GTC TTC TCC TGC-3′ (antisense), and 3) Trp6: 5′-CT ACA TTG GCG CAA AAC AG-3′ (sense) and 5′-CAC CAT ACA GAA CGT AGC CG-3′ (antisense). PCR products were ligated into pCR2.1 vectors (TA Cloning Kit, Invitrogen, San Diego, Calif.), transformed into competent cells, and screened by PCR for proper inserts. Bacterial cultures were grown for 16-18 h, and the plasmids were purified with Promega Wizard Minipreps (Madison, Wis.). Sequencing was performed by an automated fluorescence sequencer (AB1370A), and deduced amino acid alignments were carried out with the Blast software program.

### EXAMPLE I

Leakage of proteinaceous fluid from the vascular spaces into intersitial spaces and, in severe forms, into alveoli causes pulmonary edema in ARDS. Such fluid accumulation in the lung parenchyma de-oxygenates blood. Hypoxemia combined with poor tissue perfusion severely compromises organ function.

Disruption of the pulmonary endothelial cell barrier is an initiating event that promotes edema. Endothelial disruption is a regulated process occurring secondary to release of toxic oxygen radicals and proteases by white blood cells, ischemia-reperfusion injury, and/or neuro-humoral inflammatory and vasoactive mediators. These agents act as so-called first messengers to induce endothelial cell contraction and decrease cell-cell and cell-matrix tethering, resulting in gap formation between cells that forms a paracellular pathway for transfer of the proteinaceous fluid. The underlying mechanisms linking the host of first messengers to altered cell shape are unknown.

Cytosolic Ca²⁺ ([Ca²⁺]i) and adenosine 3′,5′ cyclic monophosphate (cAMP) are two intracellular signals importantly dictating endothelial cell-cell apposition, and thus permissiveness of the endothelial barrier for fluid transudation. Increased cytosolic Ca²⁺ ([Ca²⁺]i) engages the endothelial contractile apparatus to pull cells inwardly. In addition, increased [Ca²⁺]i uncouples cell-cell and cell-matrix tethering so that inward contractions produce focal gaps between cells in the vessel wall. Virtually all of these effects depend upon Ca²⁺ entry, but remarkably not a single gene product encoding a Ca²⁺ channel was originally identified in endothelial cells. Recently a Drosophila melanogaster Ca²⁺ channel called transient receptor potential, or Trp, was shown to mediate Ca²⁺ entry responsible for light perception in retina. Human homologues of this gene product were then found to be expressed in endothelial cells.

While increased [Ca²⁺]i promotes disruption of the endothelial cell barrier, increased cAMP enhances endothelial cell barrier function. Indeed, cAMP elevating agents are commonly used in clinical medicine for the treatment of inflammation. Adenylyl cyclase (the enzyme responsible for synthesis of cAMP) activators and phosphodiesterase (the enzyme responsible for the breakdown of cAMP) inhibitors have both been utilized to increase cellular cAMP content for the treatment of urticaria and asthma among other conditions. Despite an appreciation for the utility of these pharmacologic strategies in treatment of various forms of inflammation, the influence of inflammation on endothelial cell cAMP content had not been carefully investigated. Studies indicated that during inflammation cAMP levels decrease in endothelial cells, which permissively increases permeability. Interestingly, endothelial cells express a form of adenylyl cyclase that is inhibited by Ca²⁺ entry. Thus, when inflammatory first messengers stimulate Ca²⁺ entry into endothelial cells, cAMP content is reduced.

Although Trp3 and Trp6 are not SOCs (6, 46), Trp1 may form SOCs based on the following experimental evidence: 1) Trp1 and its splice variant TRPC1A increase store-operated Ca²⁺ entry when expressed in COS cells (45, 47) and 2) expression of antisense trp sequences in murine L(tk⁻) cells greatly attenuates store-operated Ca²⁺ entry evoked by Ins(1,4,5) P₃ (45). Information concerning putative functions for Trp2, -4, and -5 are lacking in the literature.

Because activation of store-operated Ca²⁺ entry is known to increase vascular permeability in isolated lungs (9, 18), thereby suggesting that pulmonary endothelial SOCs are important for regulation of endothelial barrier integrity, studies were designed to characterize the store-operated Ca²⁺ entry pathway in rat (R) pulmonary arterial endothelial cells (PAECs). The hypothesis was that a functional consequence of activating endothelial SOCs is a change in cell shape, leading to interendothelial gap formation and cytoskeletal rearrangement. To test this hypothesis, RPAECs were challenged with thapsigargin, a plant alkaloid that activates store-operated Ca²⁺ entry independent of ligand-receptor-G protein-coupled processes (40, 43), and the changes in endothelial cell shape and microfilamentous cytoskeletal arrangement were monitored. It was then determined whether RPAECs express Trp1 in order to address the possible molecular basis for the pulmonary endothelial store-operated Ca²⁺ entry pathway. Ther data indicate that store-operated Ca²⁺ entry promotes cell shape change in rat pulmonary endothelial cells expressing Trp1 and further suggest that Ca²⁺ entry through SOCs involves site-specific rearrangement of the microfilamentous cytoskeleton.

Thapsigargin Activates Store-operated Ca²⁺ Entry in RPAECs.

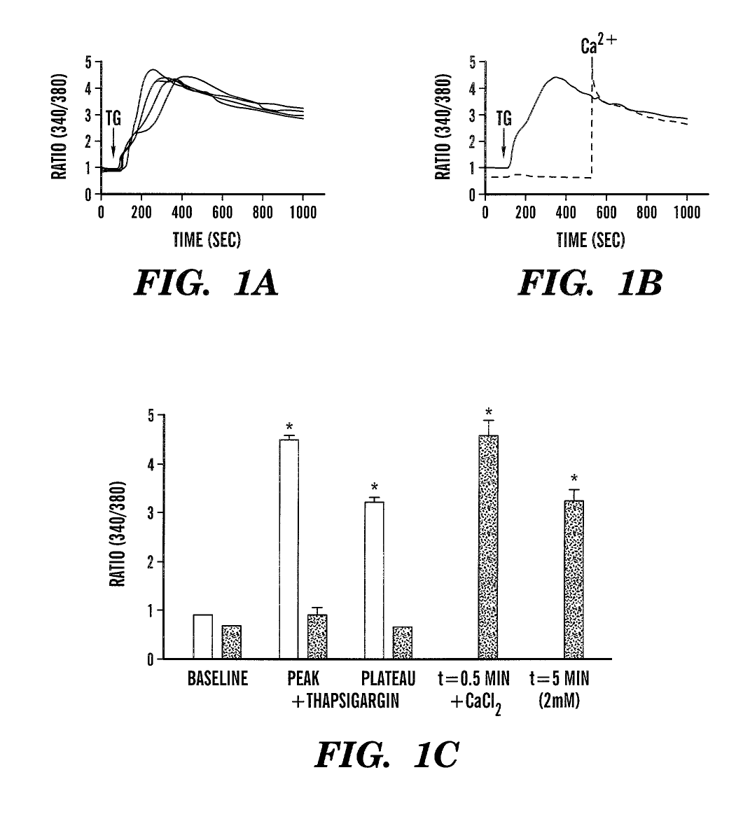

Fura 2 epifluorescence was monitored, and as shown in FIG. 1A and summarized in FIG. 1C (open bars), RPAECs incubated in 2 mM [Ca²⁺]ₒ had baseline fluorescence ratios averaging 0.91±0.02. Thapsigargin produced a gradual increase in [Ca²⁺]ᵢ to a peak level followed by a modest decline, producing a new steady state, or plateau, in [Ca²⁺]ᵢ. FIG. 1, B (dashed line) and C (solid bars), illustrates that the thapsigargin-induced response was dependent on [Ca²⁺]ₒ. When experiments were repeated in PSS containing 100 nM [Ca²⁺]ₒ, the baseline fluorescence ratio value decreased slightly, and both the peak and sustained plateau phases of the thapsigargin-induced response were significantly attenuated. Subsequence readdition of 2 mM [Ca²⁺]ₒ produced an immediate increase in [Ca²⁺]ᵢ, thereby illustrating functional store-operated Ca²⁺ entry pathways.

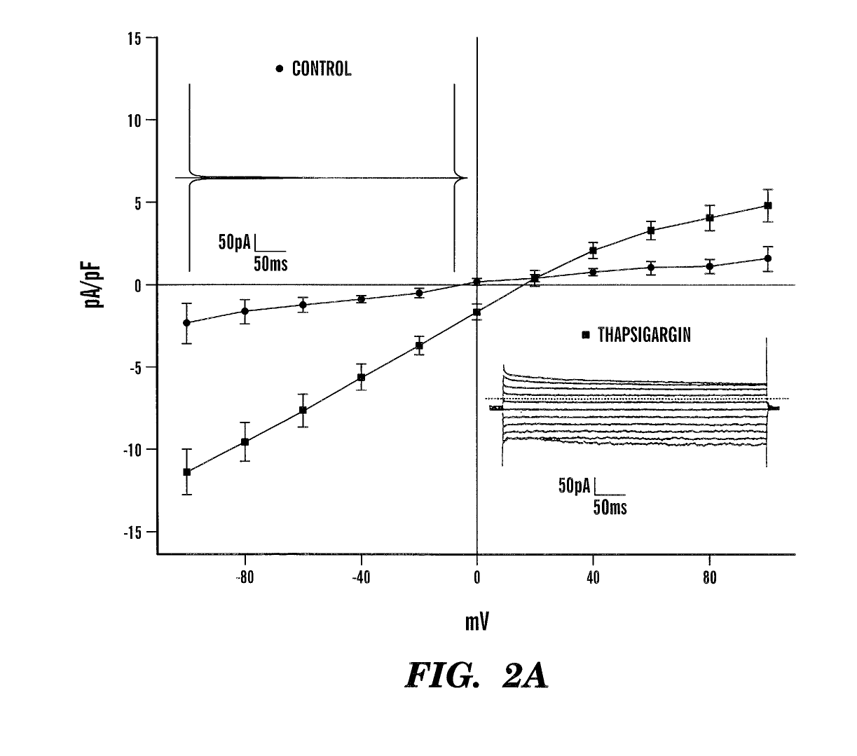

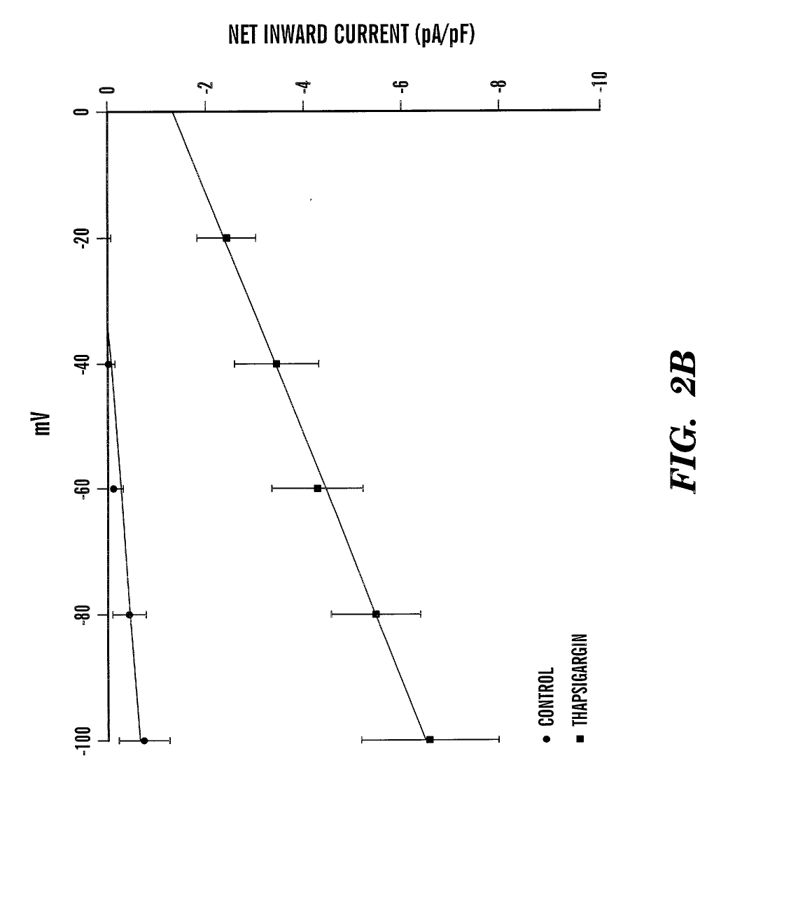

The whole cell currents from RPAECs challenged with thapsigargin were largely linear over a range of membrane potentials, although linearity was lost at about +40 mV. FIG. 2A shows current densities recorded 3-5 min after the whole cell configuration was established. Under these experimental conditions (i.e., an [Ca²⁺]ₒ-to-[Ca²⁺]ᵢ ratio of 10⁵), RPAECs had a small, net inward Ca²⁺ “leak” (control measurements without thapsigargin) calculated as 0.39±0.43 pA/pF at −80 mV (by subtraction of the outward from the inward current at each membrane potential). Thapsigargin right shifted the I-V curve and increased the current magnitude (slope conductance=1.64 nS, calculated from −100 to −20 mV without respect to cell capacitance). The net inward current stimulated by the thapsigargin was calculated as 5.45±0.90 pA/pF at −80 mV (FIG. 2B).

Store-operated Ca²⁺ Entry Evokes Endothelial Cell Shape Change and F-actin Cytoskeletal Rearrangement in RPAECs.

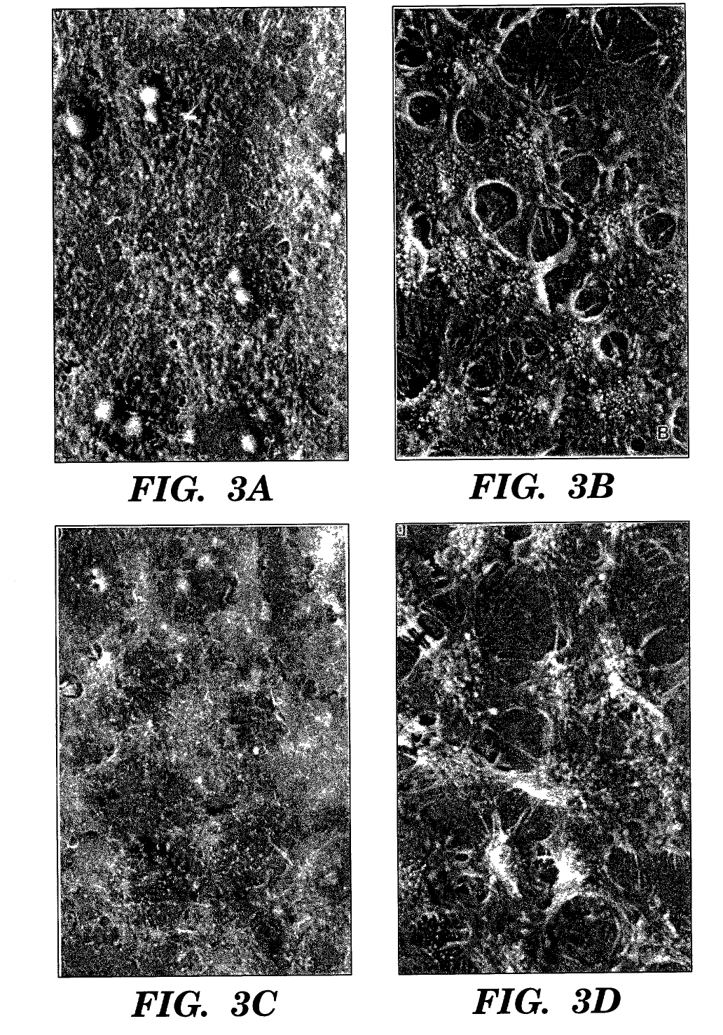

To determine a functional consequence of SOC activation in RPAECs, changes in endothelial cell shape and formation of intercellular gaps in thapsigargin-treated confluent RPAEC monolayers were assessed. Because it was determined that SOC activation was apparent 3-5 min after thapsigargin treatment, endothelial morphology at this fixed time point was studied. FIG. 3 shows scanning electron micrographs of RPAECs after different treatments. Untreated RPAECs (FIG. 3A) in 2 mM [Ca²⁺]ₒ exhibited a characteristic “cobblestone” morphology essentially devoid of intercellular gaps. Thapsigargin produced endothelial cell retraction and intercellular gap formation (FIG. 3B). The changes in endothelial cell morphology were dependent on [Ca²⁺]ₒ because RPAECs incubated in 100 nM [Ca²⁺]ₒ and challenged with thapsigargin displayed little change in morphology and a lack of interendothelial gaps (FIG. 3C). The subsequent readdition of 2 mM [Ca²⁺]ₒ had a dramatic effect on endothelial cell shape, causing pronounced cell retraction and gap formation (FIG. 3D). Thus, Ca²⁺ entry through activated SOCs sufficiently promoted endothelial cell shape alterations and interendothelial gap formation.

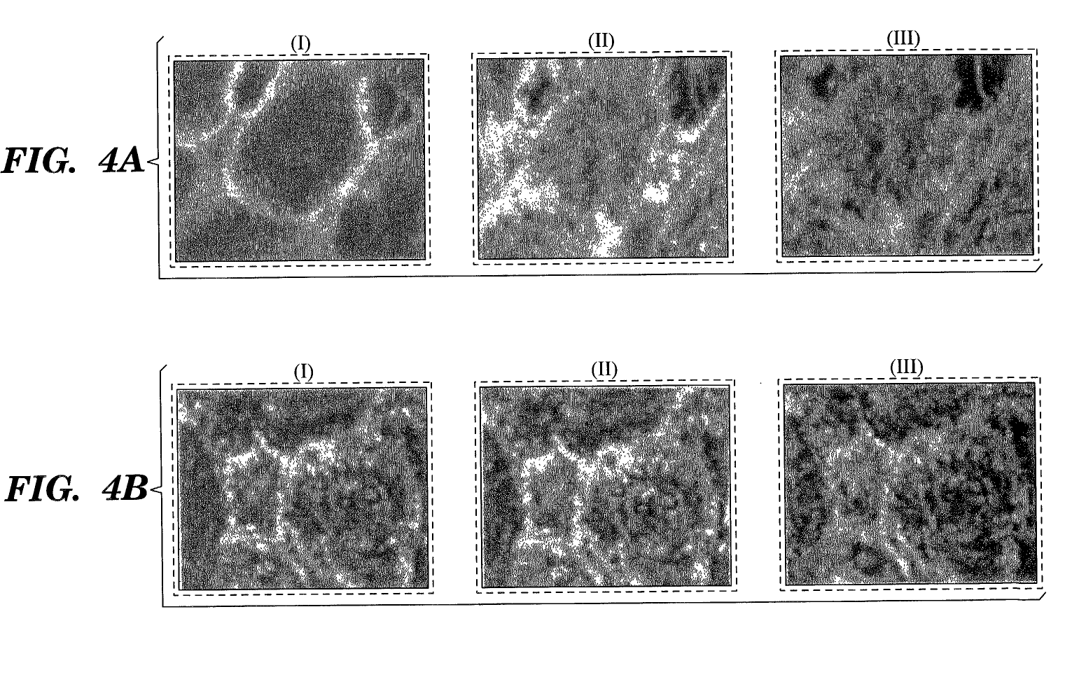

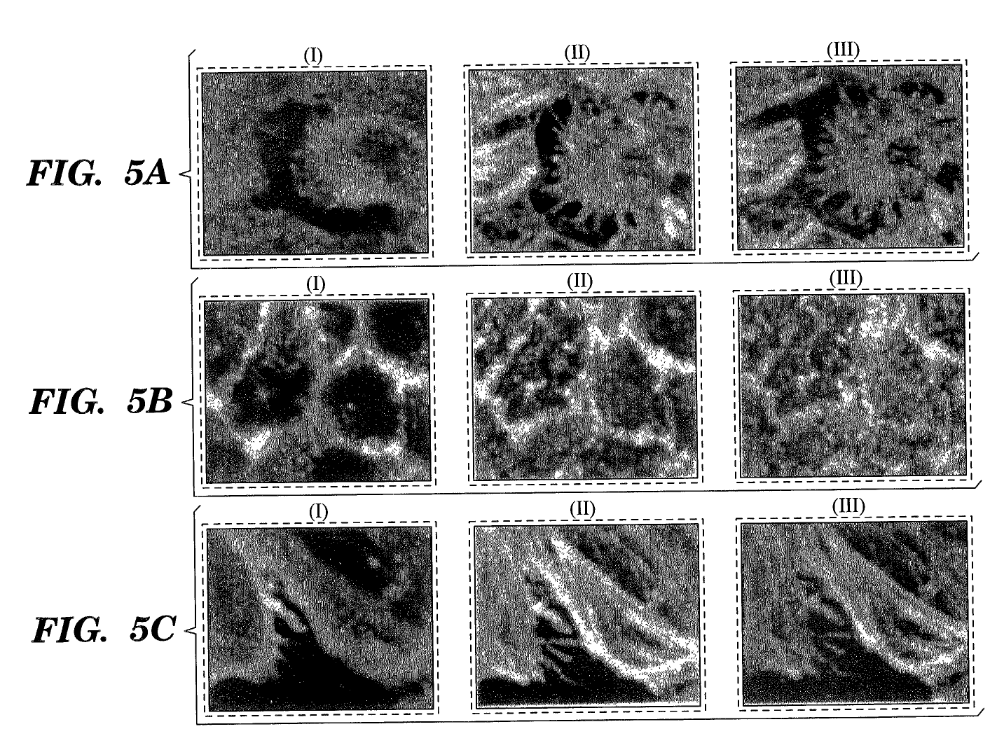

Because the actin cytoskeleton is pivotal for determining endothelial cell shape, the arrangement of F-actin in control and thapsigargin-treated RPAECs was studied. FIG. 4A shows F-actin localization in untreated RPAECs incubated with 2 mM [Ca²⁺]ₒ. Under these conditions, cells contained dense peripheral actin bands with apparent focal contact sites between cells. Some transcellular, centrally located filaments were also seen. FIG. 4B shows that incubation of RPAECs in low [Ca²⁺]ₒ alone had an effect on F-actin configuration. Diffuse, punctate F-actin staining was observed centrally in the cell, whereas densely stained focal sites at the peripheral intercellular junctions were still obvious. Thapsigargin-treated RPAECs incubated in 2 mM [Ca²⁺]ₒ (FIG. 5A) showed a decrease in peripheral F-actin density and an increase in the number and/or density of central transcellular F-actin filaments. Actin-containing projections could be seen spanning the interendothelial gaps. Thapsigargin administration to RPAECs incubated in low [Ca²⁺]ₒ (FIG. 5B) produced only modest changes in F-actin arrangements compared with incubation in low [Ca²⁺]ₒ alone. However, the subsequent readdition of 2 mM [Ca²]ₒ (FIG. 5C) produced the appearance of dense, transcellular fibers and a decrease in peripheral F-actin staining. Thus [Ca²⁺]ₒ appears to affect the localization of intracellular F-actin, and Ca²⁺ influx through activated SOCs configures the microfilamentous cytoskeleton for the alteration of cell shape.

RT-PCR Reveals the Presence of Trp1 in RPAECs.

Three specific mammalian trp gene products, Trp1, Trp3, and Trp6, were screened for because all are associated with Ca²⁺ influx into nonexcitable cell lines, although only Trp1 appears to possess the functional capacity to mediate store-operated Ca²⁺ entry. The Trp3 or Trp6 products from confluent RPAECs were not amplified. To determine whether this was a species-specific effect, RT-PCR was performed with HPAECs but likewise detected neither Trp3 nor Trp6 expression. However, both products could be amplified in rat brain, indicating that the primers were capable of amplifying these trp gene products (data not shown). In contrast, RT-PCR products for Trp1 were identified in both RPAECs and HPAECs. The RPAEC and HPAEC products were 96 and 100% homologous, respectively, to the reported nucleotide sequence for human Trp1 (Table A). The deduced amino acid alignments revealed 100% amino acid homology between both endothelial products and human Trp1 over the region studied (Table B). Thus Trp1 is present and may contribute to RPAEC SOC formation, whereas Trp3 and Trp6 likely are not expressed in the pulmonary endothelium.

Although activation of Ca²⁺ entry is sufficient to induce the interendothelial cell gap formation necessary for the transit of macromolecules and cells from blood into tissue, the mode of Ca²⁺ entry responsible for changing cell shape is unknown. Nonexcitable cells possess store-operated Ca²⁺ entry pathways. Store-operated Ca²⁺ entry is activated in response to agonist-induced stimulation of membrane phospholipases, generation of Ins(1,4,5)P₃, Ca²⁺ release from intracellular stores, and subsequent lowering of store Ca²⁺ concentrations (4, 8, 13, 16, 31, 34, 35, 41, 42). Presently, there are three prevailing questions regarding store-operated Ca²⁺ entry pathways. 1) What specific cellular functions are regulated by Ca²⁺ influx through this pathway? 2) What is the molecular identity of the membrane channels responsible for mediating store-operated Ca²⁺ entry? 3) What is the nature of the signal linking Ca²⁺ store depletion to store-operated Ca²⁺ entry? The present study addressed the first two of these three important questions.

Thapsigargin was utilized to test store-operated Ca²⁺ entry pathways because this agent produces intracellular Ca²⁺ store depletion without the confounding influences of ligand-receptor-heterotrimeric G protein activation (40, 43, 47). Fura 2-loaded RPAEC monolayers exhibited an increased [Ca²⁺]ᵢ that was dependent on Ca²⁺ influx in response to thapsigargin, thereby indicating the presence of store-operated Ca²⁺ entry pathways. To begin elucidating the electrophysiological characteristics of RPAEC SOCs, whole cell patch clamp in single cells was performed. Intracellular and extracellular patch solutions were performed to isolate thapsigargin-induced Ca²⁺ currents and determine whether thapsigargin activated a channel(s) responsible for Ca²⁺ release-activated current (I_{CRAC}) (16). However, the thapsigargin-induced current measured under these experimental conditions was not identical to I_{CRAC} because significant outward current was also measured.

It was possible that the total current measured in response to thapsigargin reflected coactivation of both a Ca²⁺-selective cation channel and an anion channel because aspartate was utilized to replace Cl⁻ in the extracellular solution, and aspartate has recently been shown to be conducted through Ca²⁺-and/or volume-activated Cl⁻ channels (29). In support of this idea, N-phenylanthranilic acid, a potent blocker of Ca²⁺-activated anion channels (27), had little effect on the inward current observed at negative voltages but strongly attenuated the outward current at positive voltages (data not shown). Thus thapsigargin may activate an anion channel capable of conducting large organic anions as previously reported in bovine pulmonary endothelium (27, 29). When the anion conductance contribution to the total thapsigargin-stimulated current is then considered, a current analogous to I_{CRAC} is apparent.

Activation of SOCs in RPAECs causes the appearance of intercellular gaps and rounding of endothelial cells. One intracellular target affected by SOC activation is plasmalemmal-associated and centrally located F-actin. It is accepted that changes in [Ca²⁺]ᵢ lead to reconfigurations of the microfilamentous cytoskeleton (21, 22, 30), although it has previously been unclear whether Ca²⁺ release from intracellular stores or Ca²⁺ influx is necessary to produce cytoskeletal changes leading to cell shape change.

Thapsigargin produced a loss of plasmalemmal F-actin staining concurrent with an increase in central F-actin staining. When store depletion alone was produced, i.e., thapsigargin in the absence of [Ca²⁺]ₒ, rearrangement of cortical actin fibers did not occur and less F-actin staining was observed centrally. Under these conditions, RPAECs did not respond to thapsigargin with a change in cell shape. The readdition of [Ca²⁺]ₒ caused morphological changes in both the peripheral (loss of dense actin staining) and centrally located (increased actin staining and transcellular filament formation) F-actin pools, indicating that Ca²⁺ influx through SOCs is sufficient to adjust the microfilament system of the cells to produce interendothelial gap formation. It is presently unclear how Ca²⁺ influx through SOCs specifically regulates the endothelial F-actin cytoskeleton, although a possible mediator of the Ca²⁺ influx-induced cytoskeletal rearrangement is Rho, a small-molecular-weight monomeric G protein, the activity of which produces actin polymerization and stress fiber formation (1, 14).

Interestingly, incubation of RPAEC monolayers in low [Ca²⁺]ₒ alone caused rearrangement of central F-actin but had no apparent effect on peripheral, or cortical, F-actin. Under these conditions, Ca²⁺ release could have been promoted because a more favorable electrochemical gradient for Ca²⁺ to leak from intracellular stores existed. Centrally located F-actin in close proximity to Ca²⁺ stores could have been affected by Ca²⁺ release but not in a manner sufficient to drive an active cell shape change. Another possibility to consider with respect to the F-actin rearrangement is that low [Ca²⁺]ₒ provided less basal Ca²⁺ influx that was somehow setting the F-actin cytoskeletal architecture.

The findings indicate that at least Trp1, but neither Trp3 nor Trp6, is expressed in pulmonary endothelial cells. It is uncertain how trp gene products may be organized in the membrane to form a functional channel, but it has been proposed that SOCs may be composed of trp homo- and/or heteromultimers (5). Because the data indicate that neither Trp3 nor Trp6 are present in rat or human pulmonary endothelial cells, the SOC is not composed of Trp1-Trp3 or Trp1-Trp6 heteromultimers.

What are the implications of the observation that SOC activation produces changes in PAEC shape? It is possible that endothelial SOCs are integral for regulating pulmonary vascular permeability responses to inflammatory mediators. Whole lung studies (9, 18) have shown that activation of SOCs alone is sufficient to produce increased vascular permeability as assessed by measures of the filtration coefficient. In addition, SOC activation promotes increased flux of macromolecules across RPAEC monolayers (19, 26). However, stimulation of the thapsigargin-sensitive store-operated Ca²⁺ entry pathway in rat pulmonary microvascular endothelial cells promotes neither increased macromolecular permeability nor changes in cell shape (19). These observations suggest that inflammatory processes involving endothelial SOC activation can produce pulmonary edema mediated by the appearance of large-vessel leak sites away from the gas-exchanging microcirculatory bed. Therefore, future studies are needed to determine whether 1) pulmonary conduit-vessel endothelium and microvascular endothelium represent distinct phenotypes having separate regulatory properties, 2) changes in conduit-vessel endothelial cell shape in situ lead to significant, function-comprising pulmonary edema, 3) the precipitating factors for increasing large-vessel and small-vessel (capillary) permeabilities are the same, and 4) interventions to alleviate pulmonary edema can be designed to selectively target conduit-vessel endothelial cells vs. microvascular endothelial cells.

The shape change elicited in response to SOC activation in RPAECs has additional importance for other endothelial-directed physiological processes such as angiogenesis and regulation of leukocyte movement. The angiogenic process requires migration of endothelial cells that, in turn, is dependent on the ability of cells to change shape and decrease their cell-to-cell and cell-to-matrix tethering (3). Inhibition of non-voltage-gated Ca²⁺ channels, presumably including SOCs, inhibits angiogenic factor-induced proliferation, migration, and tube formation of human umbilical venous endothelial cells (20), which are endothelial cells derived from conduit vessels. In addition, a study (17) has shown that human umbilical venous endothelial cell-directed regulation of leukocyte trafficking is [Ca²⁺]ᵢ dependent. Changes in endothelial cell shape and tethering that accompany neutrophil adhesion and migration require increased [Ca²⁺]ᵢ. How the increased [Ca²⁺]ᵢ occurs is not clear, but a transmembrane Ca²⁺ flux is required for certain leukocyte secretory products to increase endothelial [Ca²⁺]ᵢ (32), thereby suggesting a role for SOC-mediated Ca²⁺ entry. The data, in combination with these findings, suggest that initiation sites for angiogenesis and leukocyte diapedesis in vivo may be located in pulmonary vascular segments lined with endothelial cells possessing SOCs that regulate cell shape.

In summary, the data shows that RPAECs possess thapsigargin-activated SOCs that conduct current similar to I_{CRAC}. RPAECs respond to this mode of Ca²⁺ entry with changes in cell shape, interendothelial gap formation, and rearrangement of the F-actin cytoskeleton. Cytoskeletal rearrangement may be differentially regulated by the extracellular and intracellular Ca²⁺ pools, with Ca²⁺ influx being necessary to produce a cytoskeleton configured for cell shape change. In addition, pulmonary endothelial cells from rats (and humans) express Trp1, which may be integral for forming native SOCs in these cell types. Finally, pulmonary conduit vessel-derived endothelial SOC activation leading to interendothelial gap formation may be the basis for some forms of pulmonary edema and/or a component of angiogenesis and regulation of leukocyte trafficking to and from the vasculature.

Although preferred embodiments have been depicted and described in detail herein, it will be apparent to those skilled in the relevant art that various modifications, additions, substitutions and the like can be made without departing from the spirit of the invention and these are therefore considered to be within the scope of the invention as defined in the claims which follow.

TABLE A
sequence comparison (nucleotides 1-195) between
human Trpl (hTrpl) and RT-POR products from
human PAECs (HPAECs) and RPAECS. *Differences
between RPAEC and hTrp1 sequences.

| | |
|---|---|
| | 1 |
| hTrp1 | TCG CCG AAC GAG GTG ATG GCG CTG AAG GAT |
| HPAEC | TCG CCG AAC GAG GTG ATG GCG CTG AAG GAT |
| RPAEC | TCG CCG AAC GAG GTG ATG GCG CTG AAG GAT |
| hTrp1 | GTG CGG GAG GTG AAG GAG GAG AAT ACG CTG |
| HPAEC | GTG CGG GAG GTG AAG GAG GAG AAT ACG CTG |
| RPAEC | GTG CGA GAG GTG AAG GAG GAG AAC ACC TTG |
| | * * * * |
| hTrp1 | AAT GAG AAG CTT TTC TTG CTG GCG TGC GAC |
| HPAEC | AAT GAG AAG CTT TTC TTG CTG GCG TGC GAC |
| RPAEC | AAT GAG AAG CTT TTC TTG CTG GCG TGC GAC |
| hTrp1 | AAG GGT GAC TAT TAT ATG GTT AAA AAG ATT |
| HPAEC | AAG GGT GAC TAT TAT ATG GTT AAA AAG ATT |
| RPAEC | AAG GOT GAC TAT TAT ATG GTT AAA AAG ATT |
| hTrp1 | TTG GAG GAA AAC AGT TCA GGT GAC TTG AAC |
| HPAEC | TTG GAG GAA AAC AGT TCA GGT GAC TTG AAC |
| RPAEC | TTG GAG GAA AAC AGT TCA GGT GAC TTG AAC |
| hTrp1 | ATA AAT TGC GTA GAT GTG CTT GGG AGA AAT |
| HPAEC | ATA AAT TCC GTA GAT GTG CTT GGG AGA AAT |
| RPAEC | ATA AAT TGC GTA GAT GTG CTT GGG AGA AAT |
| hTrp1 | GCT GTT ACC ATA ACA 195 |
| HPAEC | GCT GTT ACC ATA ACA |
| RPAEC | GCT GTT ACC ATA ACA |

TABLE B
deduced amino acid sequence (amino acids 27-91)
of RPAEC and HPAEC RT-PCR products with sequence
alignment.

| | |
|---|---|
| | 27 |
| hTrp1 | S P N E V M A L K D V R E V K E E N T L N E K L. |
| HPAEC | S P N E V M A L K D V R B V K E B N T L N E K L. |
| RPAEC | S P N E V M A L K D V R E V K E E N T L N E K L. |
| hTrp1 | - F L L A C D K G D Y Y M V K K I L E - E N S S G |
| HPAEC | - F L L A C D K G D Y Y N V K K I L E - E N S S G |
| RPAEC | - F L L A C D K G D Y Y M V K K I L E - E N S S G |
| hTrp1 | D L N I N C V D V L G R N A V T I T 91 |
| HPAEC | D L N I N C V D V L G R N A V T I T |
| RPAEC | D L N I N C V D V L G R N A V T I T |

### REFERENCES

1. Aepfelbacher, M., et al., Arterioscler. Thromb. Vasc. Biol. 17:1623-1629, 1997.

2. Barnes, P., and S. Liu, Pharmacol. Rev. 47:87-131, 1995.

3. Beck, L. J., and P. D'Amore, FASEB J. 11:365-373, 1997.

4. Berridge, M., J. Physiol. (Lond.) 499:291-306, 1997.

5. Birnbaumer, L., et al., Proc. Natl. Acad. Sci. USA 93:15195-15202, 1997.

6. Boulay, G., et al., J. Biol. Chem. 272:29672-29680, 1997.

7. Braunels, U., et al., Biochem. Biophys. Res. Commun. 186:1560-1566, 1992.

8. Cabello, O., and W. Schilling, Biochem, J. 295:357-366, 1993.

9. Chetham, P., et al., Am. J. Physiol. 273 (Lung Cell. Mol. Physiol. 17):L22-L30, 1997.

10. Fabiato, A., and F. Fabiato, J. Physiol. Paris 75:463-505, 1979.

11. Friel, D., Cell 85:617-619, 1996.

12. Garcia, R. L., and W. P. Schilling, Biochem. Biophys. Res. Commun. 239:279-283, 1997.

13. Gericke, M., et al., Pflügers Arch. 422:552-557, 1993.

14. Hall, A., Science 279:509-514, 1998.

15. Hardie, R., and B. Minke, Neuron 8:643-651, 1992.

16. Hoth, M., and R. Penner, Nature 355:353-356, 1992.

17. Huang, A., et al., J. Cell Biol. 120:1371-1380, 1993.

18. Ivey, C. L., and M. I. Townsley, (Abstract) Microciruclation 4:A170, 1997.

19. Kelly, J. J., et al., Am. J. Physiol. 274 (Lung Cell. Mol. Physiol. 18):L810-L819, 1998.

20. Kohn, E., et al., Proc. Natl. Acad. Sci. USA 92:1307-1311, 1995.

21. Kuhne, W., et al., Am. J. Physiol. 264 (Heart Circ. Physiol. 33):H1599-H1608, 1993.

22. Lum, H., et al., Am. J. Physiol. 263 (Lung Cell. Mol. Physiol. 7):L219-L225, 1992.

23. Lum, H., and A. Malik, Am. J. Physiol. 267 (Lung Cell. Mol. Physiol. 11):L223-L241, 1994.

24. Mendelowitz, D., et al., Am. J. Physiol. 262 (Heart Circ. Physiol. 31):H942-H1948, 1992.

25. Montell, C., and G. Rubin, Neuron 2:1313-1323, 1989.

26. Moore, T. M., et al., (Abstract) Chest. In press.

27. Nilius, B., et al., J. Physol (Lond.) 498:381-396, 1997.

28. Nilius, B., et al., Pflügers Arch. 424:285-293, 1993.

29. Nilius, B., et al., J. Vasc. Res. 84:220-228, 1997.

30. Northover, A., Agents Actions 85:40-49, 1992.

31. Pasyk, E., et al., Am. J. Physiol. 268 (Heart Circ. Physiol. 37):H138-H146, 1995.

32. Peterson, M., et al., J. Immunol. 143:609-616, 1989.

33. Popp, R., and H. Gogelein, Biochim. Biophys. Acta 1108:59-66, 1992.

34. Putney, J., and G. Bird, Endocr. Rev. 14:610-631, 1993.

35. Schilling, W., et al., Biochem, J. 284:521-530, 1992.

36. Shasby, D., and S. Shasby, J. Appl. Physiol. 60:71-79, 1986.

37. Stevens, T., et al., In: Methods in Pulmonary Research, edited by S. Uhlis and A. E. Taylor, Basel: Birkhauser Verlag, 1997, p. 403-428.

38. Stevens, T., et al., Proc. Natl. Acad. Sci. USA 92: 2696-2700, 1995.

39. Takeda, K., and M. Klepper, Blood Vessels 27:169-183, 1990.

40. Thastrup, O., et al., Proc. Natl. Acad. Sci. USA 87:2466-2470, 1990.

41. Vaca, L., and D. Kunze, Am. J. Physiol. 264 (Heart Circ. Physiol. 33):H1319-H1322, 1993.

42. Vaca, L., and D. Kunze, Am. J. Physiol. 267 (Cell Physiol. 36):C920-C925, 1994.

43. Vaca, L., et al., Am. J. Physiol. 267 (Cell Physiol. 36):C1501-1505, 1994.

44. Ying, X., et al., Circ. Res. 79:898-908, 1996.

45. Zhu, X., et al., Cell 85:661-671, 1996.

46. Zitt, C., et al., J. Cell Biol. 138:1333-1341, 1997.

47. Zitt, C., et al., Neuron 16:1189-1196, 1996.

What is claimed is:

1. A method of decreasing inflammatory gaps in pulmonary endothelial cells, the method comprising decreasing levels of functional transient receptor potential gene product in the cells.

2. The method of claim 1 wherein decreasing levels of functional transient receptor potential gene product comprises decreasing transient receptor potential gene expression in the cells.

3. The method of claim 2 wherein decreasing transient receptor potential gene expression comprises exposing the cells to a compound which decreases transient receptor potential gene expression.

4. The method of claim 3 wherein the compound is an antisense oligonucleotide targeted to the transient receptor potential gene.

5. The method of claim 1 wherein decreasing levels of functional transient receptor potential gene product comprises exposing the cells to an inhibitor of the functional transient receptor potential gene product.

6. The method of claim 1 wherein decreasing levels of functional transient receptor potential gene product comprises exposing the cells to a compound which interferes with membrane calcium channel formation by the transient receptor potential gene product.

7. A method of treating or preventing an inflammatory condition in a subject, the method comprising administering to the subject an amount of a compound effective to decrease levels of functional transient receptor potential gene product in the cells of the subject.

8. The method of claim 7 wherein the compound decreases levels of functional transient receptor potential gene product by decreasing transient receptor potential gene expression.

9. The method of claim 8 wherein decreasing transient receptor potential gene expression comprises exposing the cells to a compound which decreases transient receptor potential gene expression.

10. The method of claim 9 wherein the compound is an antisense oligonucleotide targeted to the transient receptor potential gene.

11. The method of claim 7 wherein the compound is an inhibitor of the functional transient receptor potential gene product.

12. The method of claim 7 wherein the compound interferes with membrane calcium channel formation by the transient receptor potential gene product.

13. The method of claim 7 wherein the inflammatory condition is asthma.
